(19) United States
(12) Patent Application Publication
(10) Pub. No.: US 2026/0284363 A1
(43) Pub. Date: Sep. 24, 2026

(54) REMOVABLE ACCESS SHEATH

(71) Applicant: BOSTON SCIENTIFIC SCIMED, INC., MAPLE GROVE, MN (US)

(72) Inventors: Nicholas Lee Tassoni, Andover, MN (US); Anthony Frank Tassoni, JR., Andover, MN (US); James M. Anderson, Corcoran, MN (US); Phil Litecky, Centerville, MN (US)

(73) Assignee: BOSTON SCIENTIFIC SCIMED, INC., MAPLE GROVE, MN (US)

(21) Appl. No.: 19/566,177

(22) Filed: Mar. 13, 2026

Related U.S. Application Data
(60) Provisional application No. 63/867,193, filed on Aug. 20, 2025.
(60) Provisional application No. 63/775,508, filed on Mar. 21, 2025.

(51) Int. Cl. A61M 25/06; A61M 25/00; A61M 60/865

(52) U.S. Cl. CPC A61M 25/0668; A61M 25/0045; A61M 25/005; A61M 60/865; A61M 2210/12

(57) ABSTRACT

Example access sheaths are disclosed. An example access sheath for a percutaneous circulatory support device includes an inner liner and a plurality of support members coupled to the inner liner, wherein each support member of the plurality of support members extends at least partially around the circumference of the inner liner, and wherein each support member of the plurality of support members is spaced from one another along a longitudinal axis of the access sheath. The access sheath also includes an outer layer attached to at least a portion of each support member of the plurality of support members.

### CROSS-REFERENCE TO RELATED APPLICATIONS

This application claims the benefit of U.S. Patent Application Serial No. 63/775,508, filed Mar. 21, 2025, entitled "REMOVABLE ACCESS SHEATH”, and U.S. Patent Application Serial No. 63/867,193, filed Aug. 20, 2025, entitled “EXPANDABLE ACCESS SHEATH”, each of which are incorporated by reference herein in its entirety.

### TECHNICAL FIELD

The disclosure relates generally to medical devices and more particularly to medical devices that are adapted for use in percutaneous medical procedures.

### BACKGROUND

In some instances, performing percutaneous medical procedures may require the insertion and/or maneuvering of relatively large medical devices through a patient’s vasculature. Therefore, in some instances a relatively large vascular access sheath may be utilized to facilitate the insertion of relatively large medical devices into a patient’s vasculature. However, inserting a relatively large medical device into the vasculature through a large bore access sheath for an extended time period may, for example, lead to tissue damage (e.g., leg ischemia) due to a reduction in blood flow. Tissue damage resulting from placement of a relatively large medical device into the vasculature for an extended time period may be lessened by minimizing the size of the access sheath used to access the vessel. It may be desirable to configure a relatively large bore access sheath capable of removal for replacement with a smaller access sheath.

### SUMMARY

This disclosure provides design, material, manufacturing method, and use alternatives for medical devices. An example access sheath for a percutaneous circulatory support device includes an inner liner and a plurality of support members coupled to the inner liner, wherein each support member of the plurality of support members extends at least partially around the circumference of the inner liner, and wherein each support member of the plurality of support members is spaced from one another along a longitudinal axis of the access sheath. The access sheath also includes an outer layer attached to at least a portion of each support member of the plurality of support members.

Alternatively or additionally to any of the examples above, wherein each support member of the plurality of support members includes a first end and a second end, and wherein the first end is longitudinally offset from the second end.

Alternatively or additionally to any of the examples above, wherein the first end and the second end of each respective support member are circumferentially spaced from one another to define a first preferential separation region extending along the longitudinal axis, and wherein the access sheath is configured to separate along the first preferential separation region.

Alternatively or additionally to any of the examples above, wherein the first preferential separation region includes a plurality of perforations, and wherein the plurality of perforations extend along the longitudinal axis of the access sheath.

Alternatively or additionally to any of the examples above, wherein at least a portion of each of the plurality of perforations extends radially inward along the first preferential separation region.

Alternatively or additionally to any of the examples above, wherein each support member of the plurality of support members is configured to shift from a first position to an expanded position.

Alternatively or additionally to any of the examples above, wherein each of the support member of the plurality of support members is configured to self-bias to the expanded position.

Alternatively or additionally to any of the examples above, wherein the support members sequentially expand to facilitate splitting of the sheath along the first preferential separation region.

Alternatively or additionally to any of the examples above, wherein the outer layer includes a second preferential separation region circumferentially offset from the first preferential separation region.

Alternatively or additionally to any of the examples above, wherein the second preferential separation region is circumferentially offset 180 degrees from the first preferential separation region.

Alternatively or additionally to any of the examples above, wherein each support member of the plurality of support members is positioned between the inner liner and the outer layer.

Alternatively or additionally to any of the examples above, wherein at least a portion of the outer layer is coupled to the inner liner.

Another access sheath for a percutaneous circulatory support device includes an inner liner including an outer surface, a support wire coupled to the inner liner, wherein the support wire extends at least partially around the outer surface of the inner liner and an outer layer attached to at least a portion of the support wire.

Alternatively or additionally to any of the examples above, wherein the support wire extends continuously along the outer surface of the inner liner.

Alternatively or additionally to any of the examples above, wherein the support wire crosses over itself along at least a portion of the inner liner.

Alternatively or additionally to any of the examples above, wherein the support wire includes a first plurality of curved end loops extending along a longitudinal axis of the access sheath, a second plurality of curved end loops extending along the longitudinal axis of the access sheath, and wherein the first plurality of curved end loops are radially spaced from the second plurality of curved end loops to define a preferential separation region between the first plurality of curved end loops and the second plurality of curved end loops extending along the longitudinal axis of the access sheath.

Alternatively or additionally to any of the examples above, wherein the preferential separation region includes a first wall thickness and the outer layer includes second wall thickness, and wherein the first wall thickness is less than the second wall thickness.

Alternatively or additionally to any of the examples above, wherein the support wire is positioned between the inner liner and the outer layer.

Another access sheath for a percutaneous circulatory support device includes an inner liner and a plurality of radial support members coupled to the inner liner, wherein each radial support member of the plurality of radial support members extends at least partially around the circumference of the inner liner, and wherein each support member of the plurality of support members is spaced from one another along a longitudinal axis of the access sheath. The access sheath also includes a longitudinal support member extending at least partially along a longitudinal axis of the access sheath and an outer layer attached to at least a portion of each radial support members of the plurality of support members and the longitudinal support member.

Alternatively or additionally to any of the examples above, wherein each radial support member and the longitudinal support member are positioned between the inner liner and the outer layer.

The above summary is not intended to describe each disclosed embodiment or every implementation of the present disclosure. The Figures and Detailed Description, which follow, more particularly exemplify these examples.

### BRIEF DESCRIPTION OF DRAWINGS

FIG. 1 is a perspective view of an example medical device;

FIG. 2 is a perspective view of an example access sheath;

FIG. 3 is a perspective view of the distal end region of the access sheath of FIG. 2;

FIG. 4 is a perspective view of the distal end region of an example access sheath;

FIG. 5 is a cross-sectional view of an example access sheath in a first configuration;

FIG. 6 is a cross-sectional view of an example access sheath in a second configuration;

FIG. 7 is a perspective view of a portion of an access sheath shifting from a first configuration to a second configuration;

FIG. 8A is a cross-sectional view of an example access sheath;

FIG. 8B is a cross-sectional view of an example access sheath;

FIGS. 9-12 are example manufacturing steps of the access sheath of FIG. 2;

FIG. 13 is a perspective view of an example access sheath;

FIG. 14 is a cross-sectional of the access sheath of FIG. 13;

FIG. 15 is an example manufacturing step for the access sheath of FIG. 13;

FIG. 16 is a perspective view of an example access sheath;

FIG. 17 is a perspective view of an example access sheath;

FIG. 18 is a perspective view of an example access sheath;

FIG. 19 is a cross-sectional of the access sheath of FIG. 18;

FIG. 20 is an example manufacturing step for the access sheath of FIG. 18; and

FIG. 21 is a perspective view illustrating the separation of the hub member and the splitting of an example access sheath.

While the disclosure is amenable to various modifications and alternative forms, specifics thereof have been shown by way of example in the drawings and will be described in detail. It should be understood, however, that the intention is not to limit the disclosure to the particular examples described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some examples”, “other examples”, etc., indicate that the embodiment described may include one or more particular features, structures, and/or characteristics. However, such recitations do not necessarily mean that all examples include the particular features, structures, and/or characteristics. Additionally, when particular features, structures, and/or characteristics are described in connection with one embodiment, it should be understood that such features, structures, and/or characteristics may also be used connection with other examples whether or not explicitly described unless clearly stated to the contrary.

The following detailed description should be read with reference to the drawings in which similar elements in different drawings are numbered the same. The drawings, which are not necessarily to scale, depict illustrative examples and are not intended to limit the scope of the disclosure.

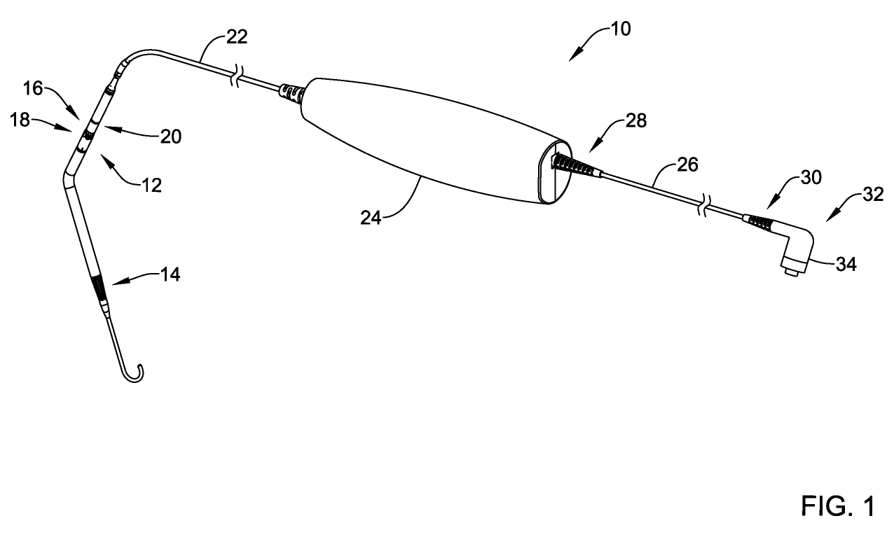

FIG. 1 shows an example percutaneous blood pump 10 (i.e., percutaneous cardiac assist device and/or circulatory support device) for connection to a medical device console (not shown). In the cases of blood pumps such as that depicted in FIG. 1, percutaneous cardiac assist devices and/or circulatory support devices support the pumping action of the heart. These devices may be positioned percutaneously into a patient’s heart, such as disposed across a valve opening such as, for example, an aortic valve. Such percutaneous blood pumps 10 assist the heart in pumping blood throughout the cardiovascular system, and the present disclosure provides uses, devices, and methods incorporating such devices as will be described herein. Such percutaneous blood pumps 10 may be connected to a medical device console for operating the blood pump and/or monitoring the operation of the blood pump and/or one or more patient parameters. However, a wide variety of medical devices are utilized to treat patients which are connected to a medical device console for operating the medical device and/or monitoring the operation of the medical device and/or one or more patient parameters. Accordingly, aspects of the present disclosure may be incorporated into a wide variety of medical devices.

Typically, a circulatory support device such as the example blood pump 10 shown in FIG. 1 includes a blood pump housing 12 including a blood inlet 14 at a blood inflow end of the blood pump housing 12 and a blood outlet 16 at a blood outflow end of the blood pump housing 12. The blood pump housing 12 may include an impeller 18 or other blood pumping device configured to convey blood through the blood pump housing 12 from the blood inlet 14 to the blood outlet 16. In some instances, the impeller 18 may be disposed within an impeller housing portion of the blood pump housing 12. The impeller 18 may be constructed of radially oriented blades protruding from a drive shaft and/or drive mechanism, for example. In some instances, the blood inlet 14 may be positioned and/or be positionable in a ventricle of the patient, while the blood outlet 16 may be positioned and/or be positionable in the aorta of the patient, such that the blood pump 10 pumps blood across the aortic valve. The impeller 18 may be driven by a motor 20 or other means of rotating the impeller 18 during operation thereof. In some instances, the motor 20 may be housed within a motor housing portion of the blood pump housing 12. In other instances, the motor 20 may be arranged at another location, if desired. For example, the motor 20 may be located on the blood pump 10 such that it remains outside the body when the blood pump 10 is in use. The motor 20 or other means of rotating the impeller 18 may be configured to rotate the impeller 18 through magnetism, electromagnetism, mechanical force, electrical force, and/or any combination or permutation of the aforementioned, such that the drive shaft and/or drive mechanism rotates the blades of the impeller 18, thereby creating a blood flow to propel blood through the blood pump housing 12 and beyond into the cardiovascular system. The blood pump 10 may include an elongate catheter shaft 22 extending proximally from the blood pump housing 12 for navigating the blood pump 10 to a desired location within the cardiovascular system of the patient. The catheter shaft 22 may extend proximally to a junction box 24, a handle, a manifold, or other structure of the blood pump 10 configured to remain exterior of the patient.

Furthermore, the blood pump 10 may include a cable 26 at a proximal end thereof having a connection interface 32 (e.g., connector) at an end thereof. The cable 26 may have a first, distal end 28 connected to the junction box 24, a handle, a manifold, or other structure of the blood pump 10, and a second, proximal end 30 having the connection interface 32 including a connector 34 that is configured to be connected to a medical device console (e.g., blood pump console). The cable 26 may include one or more of, an electrical pathway for powering the motor 20, an electrical pathway for powering one or more sensors, an electrical pathway for transmitting signals from one or more sensors associated with the blood pump 10, a fiber optic filament for transmitting signals from one or more sensors associated with the blood pump 10, or other communication conduits. In some instances, the cable 26 and associated connector 34, may be considered a single-connector cable device providing the only connection between the blood pump 10 and a medical device console controlling the blood pump 10.

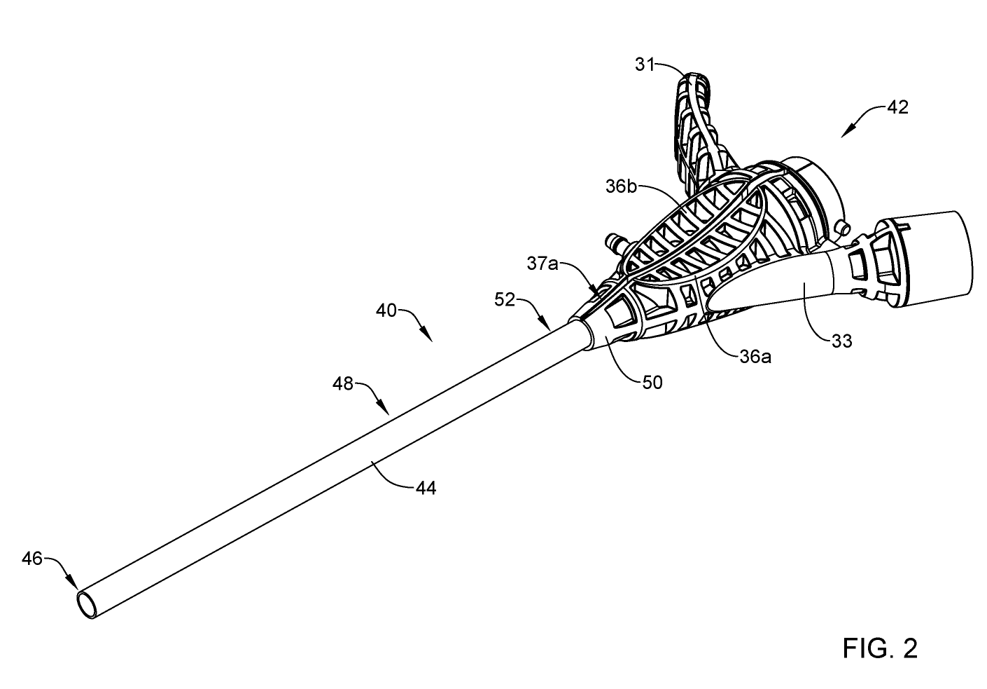

FIG. 2 is a perspective view of an example removable vascular access sheath 40 for use in providing vascular access for introducing the percutaneous blood pump of FIG. 1 into a vasculature at a vascular access site. The access sheath 40 may include a hub 42, such as a splittable hub. The access sheath may also include an elongate shaft 44 having a distal end region 46, a body portion 48 and a proximal end region 52 coupled to the hub 42.

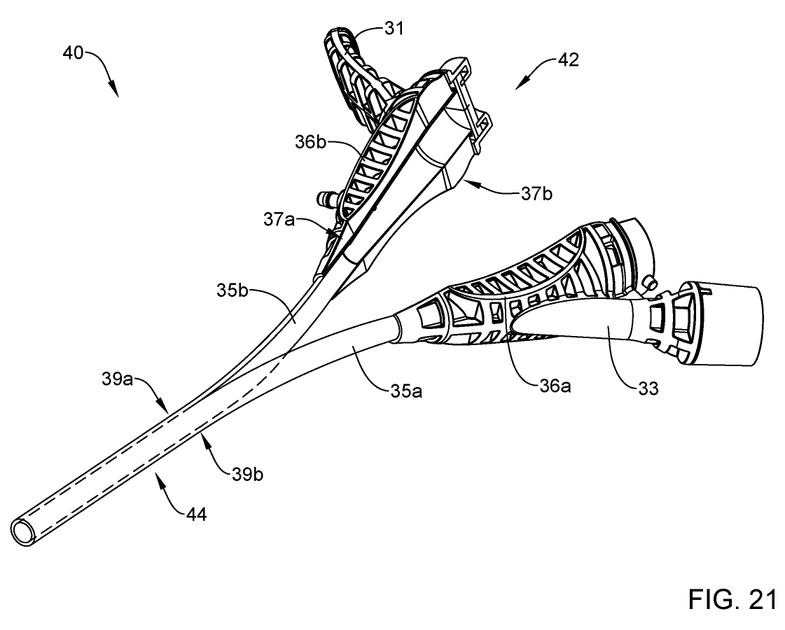

The hub 42 may also include a strain relief 50 configured to provide a transition in flexibility along the proximal end region 52 of the elongate shaft 44. The strain relief 50 may be attached to a main body of the hub 42, as will be described further herein. The splitable hub 42 may include a first hub member 36a and a second hub member 36b releasably attached together or otherwise configured to be separated from one another. In some examples, the first hub member 36a and the second hub member 36b may be releasably attached to one another via a splitable membrane. In some examples, the splitable membrane attaching the first hub member 36a and the second hub member 36b may be formed from a soft durometer material. In other examples, the first hub member 36a and the second hub member 36b may be formed as a monolithic structure configured to be selectively detached or separated from one another upon the application of a external force. For example, the hub 42 may include a frangible preferential separation region (e.g., score line, weakened area, groove, etc.) configured to be broken to separate the first hub member 36a from the second hub member 36b. The hub 42 may also include a grip member 31 and a side port 33. In some examples, the grip member 31 and/or the side port 33 may be configured to permit a user to grasp each of the grip member 31 and/or the side port 33 to separate the first hub member 36a and the second hub member 36b. In some instances, the grip member 31 may extend at an acute angle from the first hub member 36a and the side port 33 may extend at an acute angle from the second hub member 36b. The side port 33 may provide access to one or more lumens extending through the elongate shaft 44. Further, FIG. 2 illustrates that the hub 42 may include a first hub seam 37a and a second hub seam 37b (hidden from view in FIG. 2 but shown in FIG. 21), the second hub seam 37b being positioned approximately 180 degrees from the first hub seam 37a, along which the hub 42 may separate.

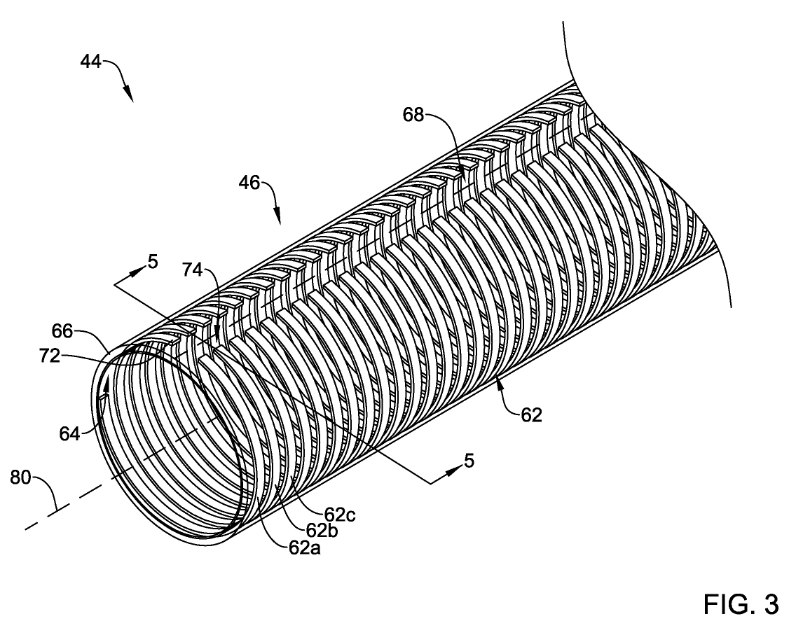

FIG. 3 illustrates the distal end region 46 of the example removeable access sheath 40 (e.g., delivery sheath, access sheath, etc.). FIG. 3 illustrates that the elongate shaft 44 of the access sheath 40 may include a plurality of reinforcing members 62 aligned along the elongate shaft 44 from the proximal end region 52 to the distal end region 46 of the elongate shaft 44. Further, it can be appreciated from FIG. 3 that each respective reinforcing member 62 may be positioned adjacent to one another along a longitudinal axis 80 of the elongate shaft 44. For example, FIG. 3 illustrates three individual reinforcing members 62a, 62b, 62c positioned adjacent to one another and aligned along the longitudinal axis 80 of the elongate shaft 44. FIG. 3 further illustrates that each of the plurality of reinforcing members 62 may partially extend about the longitudinal axis 80 of the elongate shaft 44. For example, FIG. 3 illustrates that each of the respective reinforcing members 62 may extend only partially around the longitudinal axis 80 of the elongate shaft, whereby each of the plurality of reinforcing members includes a first end circumferentially spaced away from a second end. For example, FIG. 3 illustrates that the reinforcing member 62b includes a first end 72 circumferentially spaced away from a second end 74.

As will be described in greater detail below, FIG. 3 illustrates that the elongate shaft 44 may further include a lubricious liner 64 extending from the distal end region 46 to the proximal end region 52. It can be appreciated from FIG. 3 that the liner 64 may be positioned radially inward of the plurality of the reinforcing members 62. It can be appreciated that, in some examples, an outer surface of the lubricious liner 64 may contact and be attached to an inner surface of the plurality of reinforcing members 62.

Additionally, the access sheath 40 may also include an outer layer 66. Outer layer 66 may extend along (e.g., cover) both the plurality of reinforcing members 62 and the liner 64. As will be discussed in greater detail below, the plurality of reinforcing members 62 may be positioned between (e.g., sandwiched between) the lubricious liner 64 and the outer layer 66. In some examples, the outer layer 66 may be attached to both the liner 64 and the plurality of reinforcing members 62. In some examples, the proximal region of the liner 64 and/or the proximal end region of the outer layer 66 may be attached to the hub 42.

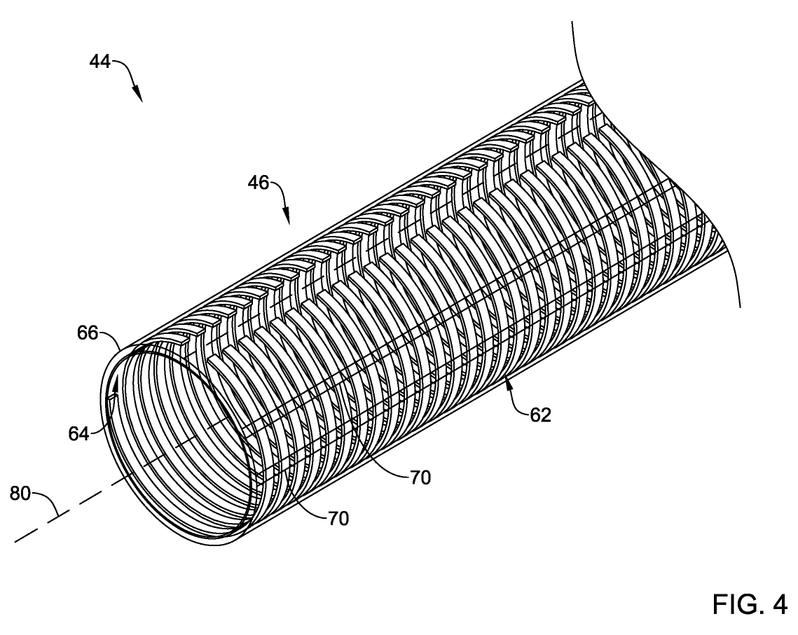

FIG. 3 further illustrates that the outer layer 66 may include a preferential separation region 68 (e.g., frangible region, splitable region, etc.). In some examples, the preferential separation region 68 may extend from the distal end region 46 to the proximal end region 52 of the elongate shaft 44. As will be discussed in greater detail herein, the preferential separation region 68 may define a longitudinally extending region of the elongate shaft 44 which is configured to split, tear, rip, etc. in response to a pull-away force being applied to the elongate shaft 44. The preferential separation region 68 may be any configuration permitting the elongate shaft 144 to be split or separated therealong. For example, the preferential separation region 68 may be a frangible region (e.g., score line, weakened area, groove, perforations, etc.) extending longitudinally along the length of the elongate shaft 44 configured to be broken. As shown in FIG. 3, the preferential separation region 68 may include a plurality of indentations (e.g., perforations) which may be aligned with one another along the central longitudinal axis 80 of the access sheath 40. Further, the preferential separation region 68 may include the region along the elongate shaft 44 in which a cutter may be utilized to cut through a wall 45 (shown in FIG. 4 and which may include the outer layer 66 and the inner layer 64) of the elongate shaft 44.

FIG. 4 illustrates that, in some examples, the access sheath 40 may also include one or more longitudinally extending reinforcing members 70. It can be appreciated that the one or more longitudinally extending reinforcing members 70 may provide reinforcing strength to the wall 45 (which may include the outer layer 66 and the liner 64) of the elongate shaft 44 in an axial direction of the elongate shaft 44.

The longitudinally extending reinforcing members 70 may extend generally parallel to the longitudinal axis 80 and perpendicular to the plurality of reinforcing members 62 described herein. In some examples, the one or more longitudinally extending reinforcing members 70 may be positioned radially inward of the reinforcing members 62. In yet other examples, the one or more longitudinally extending reinforcing members 70 may be positioned radially outward of the reinforcing members 62. In yet other examples, the one or more longitudinally extending reinforcing members 70 may be interwoven, crisscrossed, interlaced, etc. with one or more of the reinforcing members 62.

It can be appreciated that each of the one or more longitudinally extending reinforcing members 70 may be positioned between (e.g., sandwiched between) the liner 64 and the outer layer 66 described herein. In some examples, the outer layer 66 may be attached to both the liner 64 and the one or more longitudinally extending reinforcing members 70. It can be appreciated that heat may be applied to the liner 64, the spiral reinforcing members 62 and the outer layer 66 to reflow and bond the liner 64, the spiral reinforcing members 62 and the outer layer 66 together. It can be appreciated that the heating process may bond the liner 64, the spiral reinforcing members 62 and the outer layer 66 together to form the elongate shaft structure as illustrated described with respect to FIGS. 2-3. In some examples, the liner 64 may be etched or plasma treated to improve adhesion with the spiral reinforcing members 62, the outer layer 66 or both the spiral reinforcing members 62 and the outer layer 66. In some examples, the proximal region of the one or more longitudinally extending reinforcing members 70 may be attached to the hub 42.

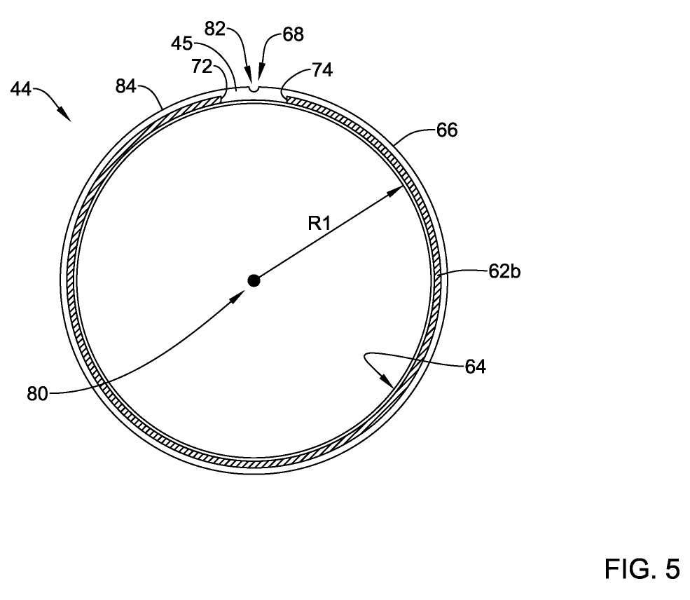

FIG. 5 illustrates an example cross-sectional view of the elongate shaft 44 taken along the line 5-5 of FIG. 3 in a first configuration. As discussed herein, FIG. 5 illustrates the reinforcing member 62b positioned between (e.g., sandwiched between) the inner lubricious liner 64 and the outer layer 66. FIG. 5 further illustrates that the inner lubricious liner 64 may extend continuously around the longitudinal axis 80 of the elongate shaft 44. FIG. 5 further illustrates that the elongate shaft 44 may include a radius R₁.

Additionally, FIG. 5 illustrates that the reinforcing member 62b extends partially around the longitudinal axis 80 of the elongate shaft 44. For example, FIG. 3 illustrates that the elongate member 62b is configured such that a first end 72 of the reinforcing member 62b is spaced away from a second end 74 of the reinforcing member 62b. In some examples, the first end and the second end of each respective reinforcing member 62 may be longitudinally offset. For example, the first end 72 of the reinforcing member 62b may be longitudinally offset from the second end 74 of the reinforcing member 62b.

As discussed herein, FIG. 5 illustrates the preferential separation region 68 positioned between the first end 72 and the second end 74 of the reinforcing member 62b. FIG. 5 illustrates that the preferential separation region 68 may include one or more regions which extend radially inward from an outer surface 84 of the outer layer 66, such as a longitudinal groove or score line. As discussed with respect to FIG. 3, the preferential separation region 68 may include a series of indentations 82 (e.g., perforations) which may be aligned with one another along the central longitudinal axis 80 of the access sheath 40. FIG. 5 illustrates an indentation 82 along the preferential separation region 68. For example, FIG. 5 illustrates a portion of the wall of the outer layer 66 is indented (e.g., extends radially inward to form a thinner wall thickness) to form the preferential separation region 68, such as a longitudinal groove or score line. In some examples, the preferential separation region 68 may include a perforation or a plurality of perforations extending longitudinally along the outer layer 66.

As discussed herein, the elongate sheath 44 may be configured to split, tear, rip, etc. in response to a pull-away (e.g., tear-away) force applied to the elongate shaft 44. For example, the access sheath 40 described herein may be utilized to insert and position a relatively large medical device (e.g., blood pump 10) within a patient. After inserting and positioning the large medical device (e.g., blood pump 10), it may be desirable to remove the access sheath 40 from medical device (e.g., remove it from the shaft 22 of the blood pump 10 which may be extending through the access sheath 40). Accordingly, a user may apply a pull-away force to the elongate shaft 44 of the access sheath 40, thereby purposefully causing the elongate shaft 44 to split, tear, rip along the preferential separation region 68 from the proximal end region 52 to the distal end region 46 of the elongate shaft 44.

Further, in some examples, each of the reinforcing members 62 of the access sheath 40 may be configured to be positioned between the liner 64 and the outer layer 66 (such as that shown in FIG. 5) under circumferential tension. In other words, during the manufacturing process, the reinforcing members 62 as shown in FIG. 5 may be placed under circumferential tension while the liner 64 and the outer layer 66 are formed around each respective reinforcing member 62, thereby maintaining each reinforcing members 62 under circumferential tension until a threshold pull-away force (e.g., tear force) is applied to the elongate shaft 44.

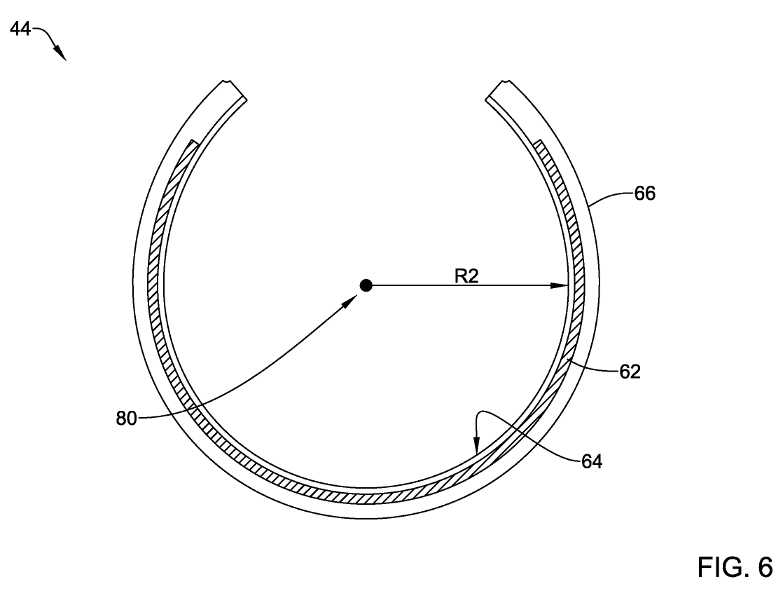

It can be appreciated that the term circumferential tension, as used herein, may describe a first configuration in which the reinforcement member 62 is held under tension in a first position (as shown in FIG. 5) and whereby each reinforcement member 62 may be bias to expand radially outward to a second position (as shown in FIG. 6, which is a cross-sectional view of an example access sheath in a second configuration) when the elongate shaft 44 splits, tears, rips, etc. thereby releasing each reinforcement member 62 from the first configuration (shown in FIG. 5) and allowing each reinforcement member 62 to expand radially outward to the second position shown in FIG. 6. FIG. 6 further illustrates that the elongate shaft 44 may include a radius R₂ in the second configuration which is greater than the radius R₁ of the elongate shaft 44 shown in the first configuration of FIG. 5. In some examples, each reinforcement member 62 may be described as having an outward radial self-bias defined by the configuration illustrated in FIG. 6. The elongate shaft 44 may split along the preferential separation region 68 when a threshold pull-away force (e.g., tear force) is applied.

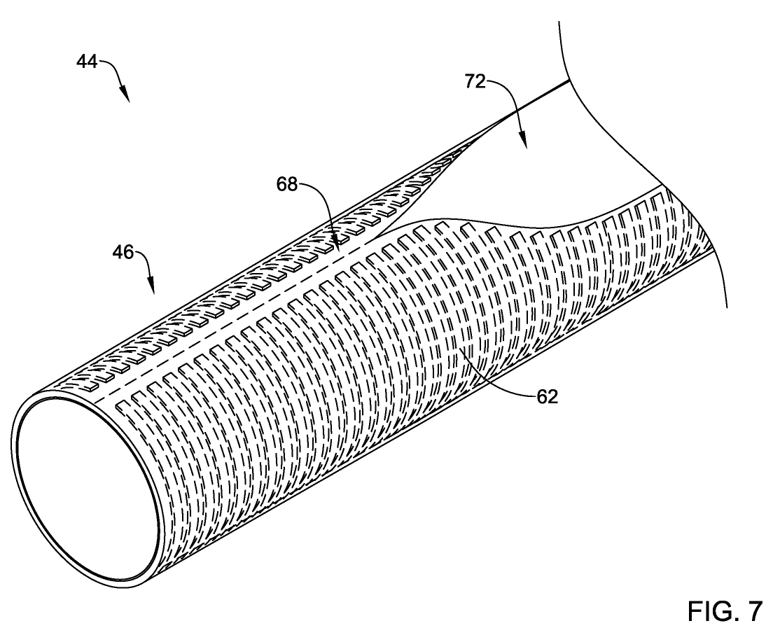

FIG. 7 illustrates a perspective view of the elongate shaft splitting from the proximal end region 52 (shown in FIG. 2) to the distal end region 46 of the elongate shaft 44. As described herein, it can be appreciated that the splitting of the elongate shaft 44 along the preferential separation region 68 may occur continuously from the proximal end region 52 to the distal end region 46 of the elongate shaft 44. However, in other examples, splitting of the elongate shaft 44 along the preferential separation region 68 may occur continuously from the distal end region 46 to the proximal end region 46 of the elongate shaft 44. In yet other examples, splitting of the elongate shaft 44 along the preferential separation region 68 may begin in the body 48 portion of the elongate shaft 44 and proceed toward the proximal end region 52, toward the distal end region 46 or toward both the proximal end region 52 and the distal end region 46.

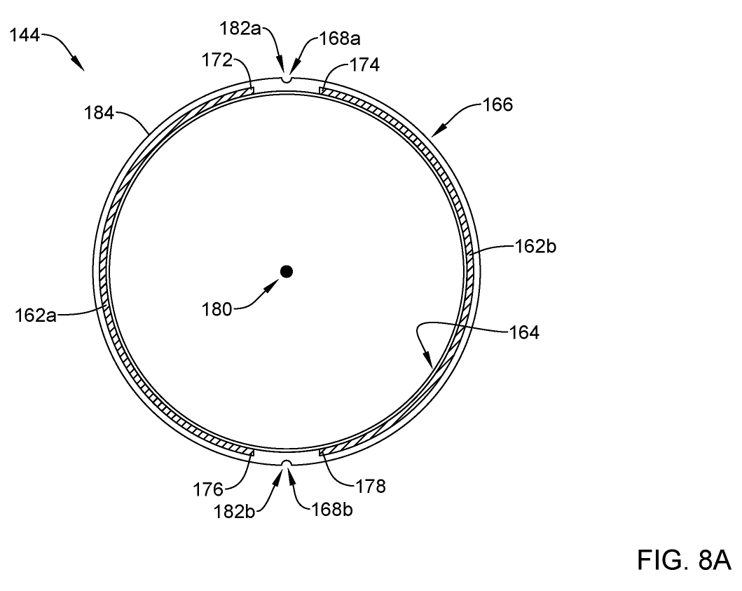

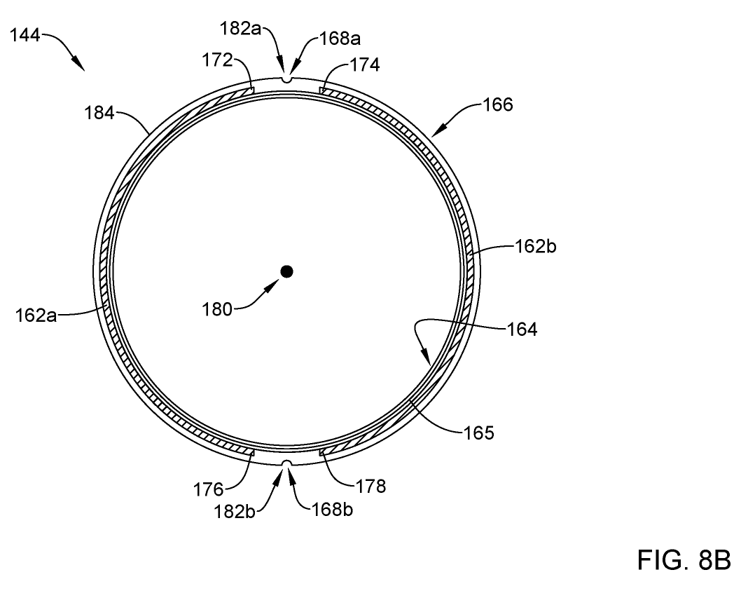

FIG. 8A illustrates an example cross-sectional view of another example elongate shaft 144. The elongate shaft 144 may be similar in form and function to the elongate shaft 44 of the access sheath 40 described herein. However, FIG. 8A illustrates that the elongate shaft 144 may include both a first reinforcing member 162a and a second reinforcing member 162b positioned between (e.g., sandwiched between) the inner lubricious liner 164 and the outer layer 166. As illustrated in FIG. 8A, the first reinforcing member 162a and the second reinforcing member 162b may be circumferentially offset from one another. FIG. 8A illustrates that the inner lubricious liner 164 may extend continuously around the longitudinal axis 180 of the elongate shaft 144.

Additionally, FIG. 8A illustrates that each of the reinforcing members 162a, 162b extend partially around the longitudinal axis 180 of the elongate shaft 144. For example, FIG. 8A illustrates that the first reinforcing member 162a and the second reinforcing member 162b are configured such that a first end 172 of the first reinforcing member 162a is spaced away from the first end 174 of the second reinforcing member 162b and a second end 176 of the first reinforcing member 162a is spaced away from the second end 178 of the second reinforcing member 162b.

Like that described herein with respect to the elongate shaft 44, FIG. 8A illustrates that the elongate shaft 144 may include a first preferential separation region 168a positioned between the first end 172 of the first reinforcing member 162a and the first end 174 of the second reinforcing member 162b. Additionally, FIG. 8A illustrates that the elongate shaft 144 may include a second preferential separation region 168b positioned between the second end 176 of the first reinforcing member 162a and the second end 178 of the second reinforcing member 162b. The preferential separation regions 168a, 168b may be any configuration permitting the elongate shaft 144 to be split or separated therealong. For example, the preferential separation regions 168a, 168b may be frangible regions (e.g., score line, weakened area, groove, perforations, etc.) extending longitudinally along the length of the elongate shaft 144 configured to be broken to separate the elongate shaft 144 into multiple pieces. FIG. 8A illustrates that each of the first preferential separation region 168a and the second preferential separation region 168b may include one or more regions which extend radially inward from an outer surface of the outer layer 166 to thereby form a thinned wall section (e.g. score line, groove, etc.). As discussed with respect to FIG. 3, the preferential separation region 168a may include a series of indentations (e.g., perforations) 182a which may be aligned with one another along the central longitudinal axis 180 of the elongate shaft 144. Similarly, the preferential separation region 168b may include a series of indentations (e.g., perforations) 182b which may be aligned with one another along the central longitudinal axis 180 of the elongate shaft 144. In some examples, the preferential separation regions 168a, 168b may each include perforated preferential separation regions whereby each preferential separation region 168a, 168b includes a plurality of perforations extending longitudinally along the outer layer 166.

As discussed herein, the elongate sheath 144 may be configured to split, tear, rip, etc. in response to a pull-away (e.g., tear-away) force applied to the elongate shaft 144. Like that described with respect to the elongate shaft 44, a user may apply a pull-away force to the elongate shaft 144, thereby purposefully causing the elongate shaft 144 to split, tear, rip along both the first preferential separation region 168a and the second preferential separation region 168b along the length of the elongate shaft 144.

Further, in some examples, each of the reinforcing members 162a, 162b may be configured to be positioned between the liner 164 and the outer layer 166 (such as that shown in FIG. 8A) under circumferential tension. In other words, during the manufacturing process, the reinforcing members 162a, 162b as shown in FIG. 8A may be placed under circumferential tension while the liner 164 and the outer layer 166 are formed around each respective reinforcing member 162a, 162b, thereby maintaining each reinforcing member 162a, 162b under circumferential tension until a threshold pull-away force (e.g., tear force) is applied to the elongate shaft 144.

FIG. 8B illustrates another example cross-sectional view of the example elongate shaft 144. The cross-sectional view of FIG. 8B of elongate shaft 144 may be similar in form and function to the cross-sectional view of FIG. 8A and the elongate shaft 44 of the access sheath 40 described herein. However, FIG. 8B illustrates that the elongate shaft 144 may include both a first reinforcing member 162a and a second reinforcing member 162b positioned between (e.g., sandwiched between) an inner layer 165 and the outer layer 166 (FIG. 8B further illustrates the inner lubricious liner 164 positioned radially inward of the inner layer 165). Thus, the inner lubricious liner 164 may define the lumen extending through the elongate shaft 144, the inner layer 165 may be positioned radially outward of and circumferentially surround the inner lubricious layer 164, the first and second reinforcing members 162a, 162b may be positioned radially outward of and circumferentially surround the inner layer 165, and the outer layer 166 may be positioned radially outward of and circumferentially surround the first and second reinforcement members 162a, 162b. As illustrated in FIG. 8B, the first reinforcing member 162a and the second reinforcing member 162b may be circumferentially offset from one another. FIG. 8B illustrates that both the inner layer 165 and the inner lubricious liner 164 may extend continuously around the longitudinal axis 180 of the elongate shaft 144.

Additionally, FIG. 8B illustrates that each of the reinforcing members 162a, 162b extend partially around the longitudinal axis 180 of the elongate shaft 144. For example, FIG. 8B illustrates that the first reinforcing member 162a and the second reinforcing member 162b are configured such that a first end 172 of the first reinforcing member 162a is spaced away from the first end 174 of the second reinforcing member 162b and a second end 176 of the first reinforcing member 162a is spaced away from the second end 178 of the second reinforcing member 162b.

Like that described herein with respect to the elongate shaft 44, FIG. 8B illustrates that the elongate shaft 144 may include a first preferential separation region 168a positioned between the first end 172 of the first reinforcing member 162a and the first end 174 of the second reinforcing member 162b. Additionally, FIG. 8A illustrates that the elongate shaft 144 may include a second preferential separation region 168b positioned between the second end 176 of the first reinforcing member 162a and the second end 178 of the second reinforcing member 162b. The preferential separation regions 168a, 168b may be any configuration permitting the elongate shaft 144 to be split or separated therealong. For example, the preferential separation regions 168a, 168b may be frangible regions (e.g., score line, weakened area, groove, perforations, etc.) extending longitudinally along the length of the elongate shaft 144 configured to be broken to separate the elongate shaft 144 into multiple pieces. FIG. 8B illustrates that each of the first preferential separation region 168a and the second preferential separation region 168b may include one or more regions which extend radially inward from an outer surface of the outer layer 166 to thereby form a thinned wall section (e.g. score line, groove, etc.). As discussed with respect to FIG. 3, the preferential separation region 168a may include a series of indentations (e.g., perforations) 182a which may be aligned with one another along the central longitudinal axis 180 of the elongate shaft 144. Similarly, the preferential separation region 168b may include a series of indentations (e.g., perforations) 182b which may be aligned with one another along the central longitudinal axis 180 of the elongate shaft 144. In some examples, the preferential separation regions 168a, 168b may each include perforated preferential separation regions whereby each preferential separation region 168a, 168b includes a plurality of perforations extending longitudinally along the outer layer 166.

As discussed herein, the elongate shaft 144 may be configured to split, tear, rip, etc. in response to a pull-away (e.g., tear-away) force applied to the elongate shaft 144. Like that described with respect to the elongate shaft 44, a user may apply a pull-away force to the elongate shaft 144, thereby purposefully causing the elongate shaft 144 to split, tear, rip along both the first preferential separation region 168a and the second preferential separation region 168b along the length of the elongate shaft 144.

Further, in some examples, each of the reinforcing members 162a, 162b may be configured to be positioned between the liner 164 and the outer layer 166 (such as that shown in FIG. 8B) under circumferential tension. In other words, during the manufacturing process, the reinforcing members 162a, 162b as shown in FIG. 8B may be placed under circumferential tension while the liner 164 and the outer layer 166 are formed around each respective reinforcing member 162a, 162b, thereby maintaining each reinforcing member 162a, 162b under circumferential tension until a threshold pull-away force (e.g., tear force) is applied to the elongate shaft 144.

In other examples of the shaft 144 shown in FIGS. 8A-8B, however, the reinforcing members 162a, 162b may not be placed under circumferential tension during manufacturing. In such examples, the reinforcing members 162a,162b may be positioned between the liner 164 and the outer layer 166 (in the example of 8A) or between the inner layer 165 and the outer layer 166 (in the example of FIG. 8B) without being biased to separate from one another, which may permit the shaft 144 to maintain a substantially circular cross-sectional shape and reduce or prevent ovalization of the sheath 144 during manufacturing, for example.

Additionally, in some examples, the preferential separation regions 168a, 168b of the shaft 144 may not include indentations or perforations. Instead, the preferential separation regions 168a, 168b may be defined by a region of one or more of the outer layer 166, the inner layer 165 (in the example of FIG, 8B) and the liner 164 having a reduced wall thickness and/or a reduced outer diameter relative to adjacent portions of the elongate shaft 144, thereby defining a region configured to preferentially separate when a threshold pull-away force is applied.

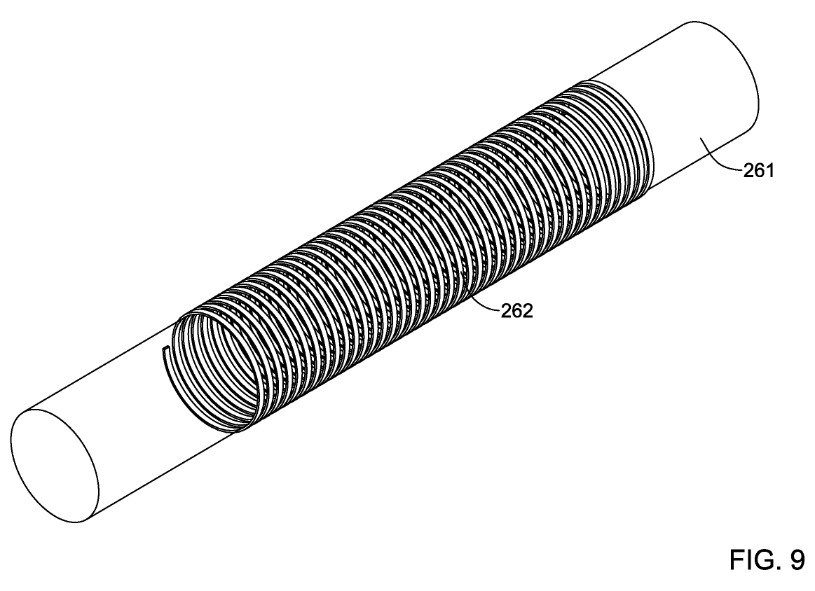

FIGS. 9-12 illustrate an example manufacturing process for constructing the elongate shaft 44 of the access sheath 40 described herein.

FIG. 9 illustrates that an example manufacturing step may include positioning a spiral or helical reinforcing member 262 on a cylindrical mandrel 261. The spiral reinforcing member 262 may be formed from a variety of materials, including metallic materials (e.g., stainless steel, tungsten, nickel-titanium, etc.), polymeric materials (e.g., polyimide, polyethylene naphthalate, liquid crystal polymers, polyether ether ketone, etc.), composite metallic and polymeric materials, ceramic materials, etc. A non-exhaustive list of materials which may be utilized to construct the spiral reinforcing member 262 is disclosed herein.

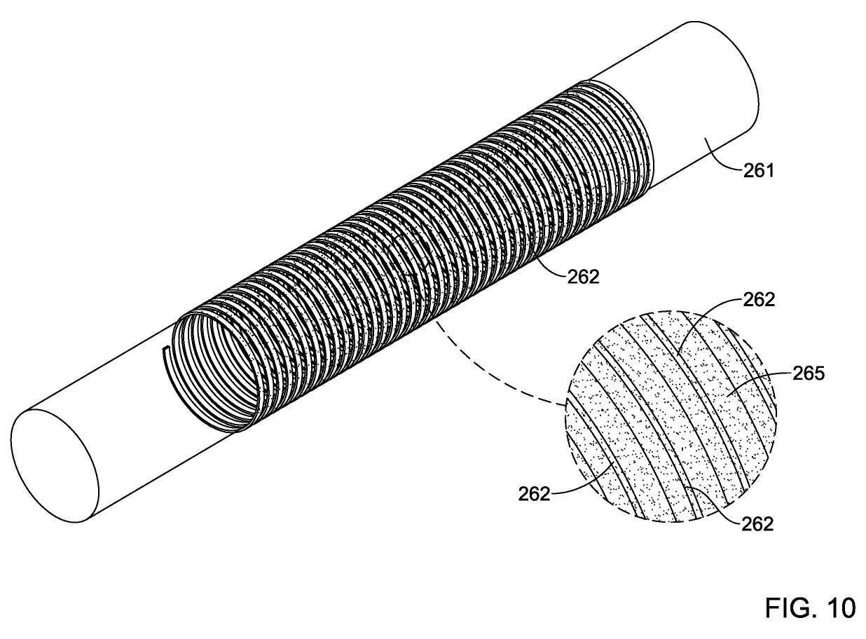

FIG. 10 illustrates another example manufacturing step for constructing the access sheath 10 described herein. FIG. 10 illustrates that the spiral reinforcing member 262 may be coated with a thin polymer 265 (shown in the detailed view of FIG. 10), whereby the thin polymer 265 is configured to effectively bond the coils of the spiral reinforcing member 262 together and in a fixed position relative to one another. In some examples, the thin polymer 265 may include 55D Pebax^{®} or other similar material.

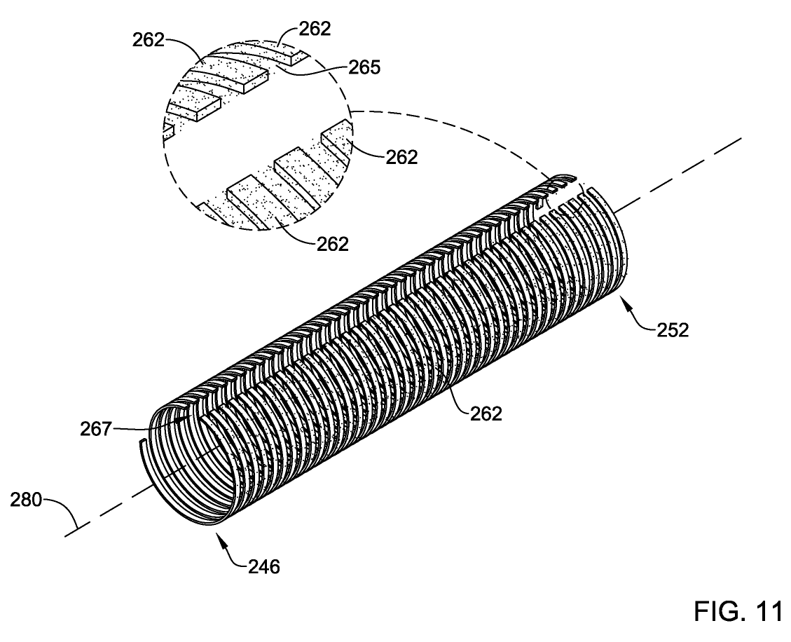

FIG. 11 illustrates another example manufacturing step for constructing the access sheath 40 described herein. FIG. 11 illustrates that a laser or other mechanical cutting method may be utilized to make a cut 267 along the longitudinal axis of the bonded spiral reinforcing member 262 shown in FIG. 10. The cut 267 may extend from a distal end region 246 of the spiral reinforcing member 262 to a proximal end region 252 of the spiral reinforcing member 262. It can be appreciated that the cutting process as described in this step modifies the single spiral reinforcing member 262 into a plurality of individual reinforcing members 62 (shown in FIG. 3).

In some examples, it may be desirable to make multiple cuts in the spiral reinforcing member 262. For example, when constructing the elongate shaft illustrated and described with respect to FIG. 8, a first cut and a second cut may be formed in the spiral reinforcing member 262, whereby each of the first cut and the second cut are offset from one another approximately about 120 to about 180 degrees. It can be further appreciated that, when constructing the elongate shaft illustrated and described with respect to FIG. 8, the two halves of the spiral reinforcing member 262 that are formed when making two cuts of the spiral reinforcing member 262 may be temporarily held together via one or more tacked regions and/or one or more sacrificial bonding regions which extend beyond the distal end region 246 and the proximal end region 252 and are cut off during subsequent manufacturing steps.

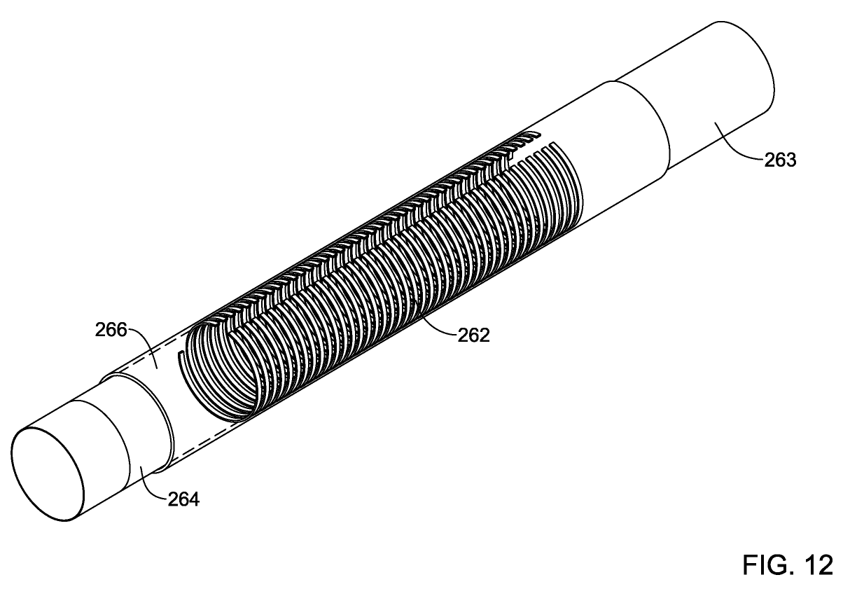

FIG. 12 illustrates another example manufacturing step for constructing the access sheath 10 described herein. FIG. 12 illustrates that the bonded and cut spiral reinforcing member 262 described with respect to FIG. 11 may be positioned over a lubricious liner 264 which has been positioned along a mandrel 263.

It can be appreciated that, in some examples, the mandrel 263 may include an outer diameter which is smaller than the resting inner diameter of the spiral reinforcing member 262. Accordingly, the spiral cut reinforcing member 262 may be positioned over the liner 264 under tension. In other words, the spiral reinforcing member 262 may be radially compressed along the liner 264 (which is positioned along the mandrel 263) such that the spiral reinforcing member 262 is placed under circumferential tension. As described herein, when placed under circumferential tension, the spiral reinforcing member 262 may be held under tension in a first position whereby the reinforcement member 262 may be bias to expand radially outward to a second position when released from a constraint (such as when the elongate shaft 44 splits, tears, rips, etc. as described herein).

Further, an outer layer 266 may also be positioned over the bonded and cut spiral reinforcing member 262 while the spiral reinforcing member 262 is under circumferential tension. FIG. 12 illustrates a manufacturing step in which the lubricious liner 264, the bonded and cut spiral reinforcing member 262 and the outer layer 266 may be stacked on top of one another along a mandrel 263. Further, it can be appreciated that heat may be applied to the lubricious liner 264, the spiral reinforcing member 262 and the outer layer 266 to reflow and bond the lubricious liner 264, the spiral reinforcing member 262 and the outer layer 266 together to form the elongate shaft structure as illustrated and described with respect to FIGS. 2-3. In some examples, the lubricious liner 264 may be etched or plasma treated to improve adhesion with the spiral reinforcing member 262, the outer layer 266 or both the spiral reinforcing member 262 and the outer layer 266.

Additionally, in some examples, the material used to construct the outer layer 266 may be the same material utilized to effectively bond the coils of the spiral reinforcing member 262 in a fixed position relative to one another (as described with respect to FIG. 10). Accordingly, when the heating and reflow process is performed (as described with respect to FIG. 12), the material of the outer layer 266 may effectively combine with the polymer used to bond the coils of the spiral reinforcing member 262 in a fixed position relative to one another, thereby forming a monolithic outer layer 66 (shown in FIG. 3) formed of a homogenous polymer. It can be further appreciated that the heating and reflow process performed (as described with respect to FIG. 12) may chemically or mechanically bond the homogenous outer layer 66 with the liner 64 (shown in FIG. 3). It can further be appreciated that after the heating process described with respect to FIG. 12 is complete, the plurality of individual reinforcing members 62 will be positioned between the outer layer 66 and the liner 64 as described herein.

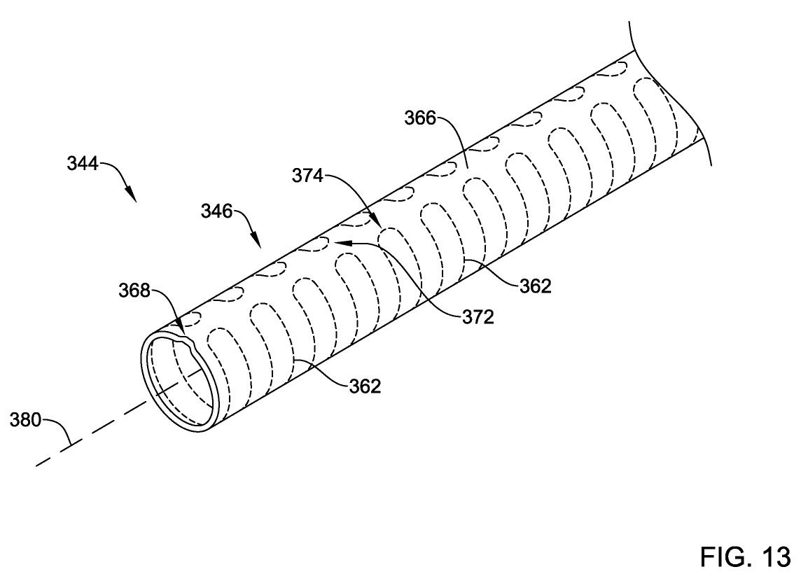

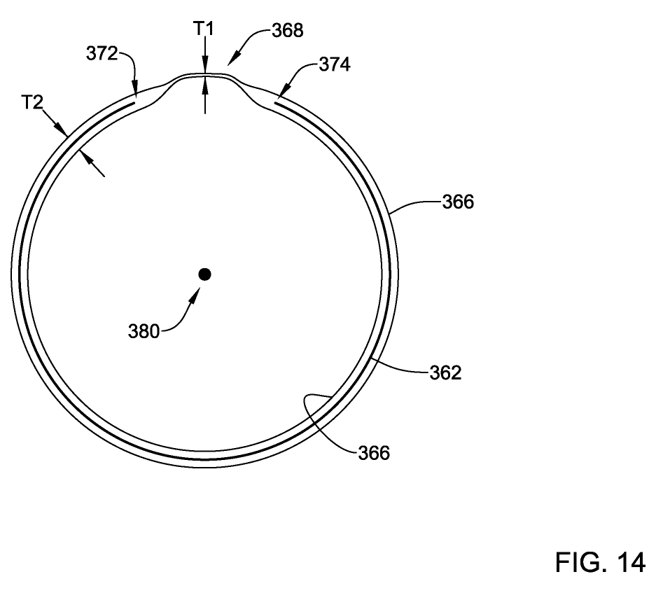

FIG. 13 illustrates another example elongate shaft 344 of the access sheath 40 (shown in FIG. 2) and FIG. 14 is a cross-sectional view of the access sheath of FIG. 13. The elongate shaft 344 may be similar in form and function to the elongate shaft 44 described herein. However, FIG. 13 illustrates that the elongate shaft 344 may include a reinforcing wire 362 positioned in the wall 366 of the elongate shaft 344, whereby the reinforcing wire 362 extends continuously along the elongate shaft 344 from the proximal end region to the distal end region 346 of the elongate shaft 344. FIG. 13 further illustrates that the reinforcing wire 362 may extend only partially about a longitudinal axis 380 (e.g., partially in a circumferential direction about the longitudinal axis 380), whereby the reinforcing wire 362 includes a row of first looped ends 372 circumferentially spaced away from a row of second looped ends 374. In some examples, the wall 366 of the elongate shaft 344 may be formed from one or more layers. For example, the elongate shaft 344 may include a lubricious inner layer or inner liner. In other examples, the wall 366 of the elongate shaft may be coated with a hydrophilic or hydrophobic lubricious coating.

FIG. 13 further illustrates that the elongate shaft 344 may include a preferential separation region 368. In some examples, the preferential separation region 368 may extend from the distal end region 346 to the proximal end region of the elongate shaft 344. As will be discussed in greater detail herein, the preferential separation region 368 may define a longitudinally extending region of the elongate shaft 344 which is configured to split, tear, rip in response to a pull-away force being applied to the elongate shaft 344. Further, FIG. 14 illustrates that a portion of the wall 366 of the elongate sheath 344 may be tapered about the circumference of the elongate shaft 344 to form a reduced thickness region of the wall 366 which defines the preferential separation region 368. For example, FIG. 14 illustrates the portion of the wall 366 which forms the preferential separation region 368 may have a wall thickness T_{1,} while the portion of the wall 366 which is circumferentially spaced away from the preferential separation region 368 may have a wall thickness T₂, whereby T₂ is greater than T₁.

Further, in some examples, the material used to construct the preferential separation region 368 (e.g., the material used to construct the longitudinally extending region of the elongate shaft 344 which is configured to split, tear, rip in response to a pull-away force being applied to the elongate shaft 344) may include materials which may be configured to enhance splitting or “radial compliance.” The properties (e.g., durometer, modulus, composite of elements, etc.) of these materials may enhance splitting or radiopacity, for example. In some examples, these materials may include barium, bismuth, tungsten, or other similar materials.

FIG. 14 also illustrates the reinforcing wire 362 positioned within the wall 366 of the elongate shaft 344, whereby the wire reinforcing member 362 extends only partially about the longitudinal axis 380 (e.g., partially in a circumferential direction about the longitudinal axis 380). Further, FIG. 14 illustrates the row of first looped ends 372 spaced apart from the row of second looped ends 374. Further, FIG. 14 illustrates the reduced thickness region of the wall 366 which defines the preferential separation region 368 positioned between the row of first looped ends 372 and the row of second looped ends 374.

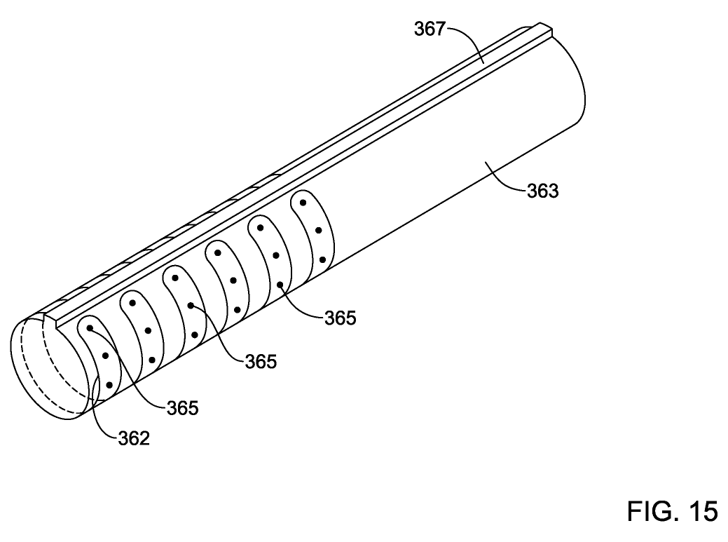

FIG. 15 illustrates an example manufacturing step for constructing the elongate sheath 344 described herein. FIG. 15 illustrates that the reinforcing wire 362 may be wound along a mandrel 363, whereby the mandrel 363 includes a plurality of pins 365 arranged to form the continuously wound reinforcing wire 362 illustrated in FIG. 13. It can be appreciated that the arrangement of the pins 365 configures the reinforcing wire 362 to extend only partially about the longitudinal axis 380 (e.g., partially in a circumferential direction about the longitudinal axis 380) and form the row of first looped ends 372 spaced apart from the row of second looped ends 374, as illustrated in FIG. 13. Further, it can be appreciated that that mandrel 363 may include a spine 367 that permits the formation of the reduced thickness region of the wall 366 which defines the preferential separation region 368, whereby the preferential separation region 368 is positioned between the row of first looped ends 372 and the row of second looped ends 374. It can be appreciated that a polymer material may be applied to the manufacturing assembly illustrated in FIG. 15 to form the wall 366 (including the reinforcing wire 362 positioned therein). The mandrel 363 may then be removed to form the elongate shaft 344 described herein.

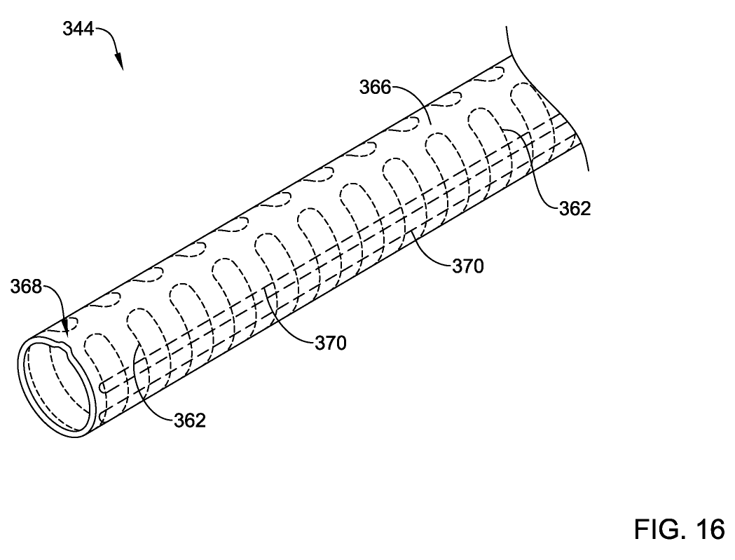

FIG. 16 illustrates another example embodiment of the elongate shaft 344 illustrated and described with respect to FIG. 13. Further, FIG. 16 illustrates that, in some examples, the elongate shaft 344 may also include one or more longitudinally extending reinforcing wires 370. It can be appreciated that the one or more longitudinally extending reinforcing wires 370 may provide reinforcing strength to the wall of the elongate shaft 344 in an axial direction of the elongate shaft 344. It can be further appreciated that the one or more longitudinally extending reinforcing wires 370 may enhance radial strength, radial compression, kink resistance and/or buckling resistance to the wall of the elongate shaft 344.

The longitudinally extending reinforcing wires 370 may extend generally parallel to the longitudinal axis 380 and perpendicular to the reinforcing wire 362 described herein. In some examples, the longitudinally extending reinforcing wires 370 may wind around the longitudinal axis 380 and engage the reinforcing wires 362 described herein. In some examples, the one or more longitudinally extending reinforcing wire members 370 may be positioned radially inward of the reinforcing wire 362. In yet other examples, the one or more longitudinally extending reinforcing wires 370 may be positioned radially outward of the reinforcing wire 362. In yet other examples, the one or more longitudinally extending reinforcing wires 370 may be interwoven, crisscrossed, interlaced, welded, joined, bonded, etc. with the reinforcing wire 362.

FIG. 16 further illustrates that the elongate shaft 344 may include a preferential separation region 368. In some examples, the preferential separation region 368 may extend from the distal end region 346 to the proximal end region of the elongate shaft 344. As discussed with respect to FIG. 13, the preferential separation region 368 may define a longitudinally extending region of the elongate shaft 344 which is configured to split, tear, rip in response to a pull-away force being applied to the elongate shaft 344.

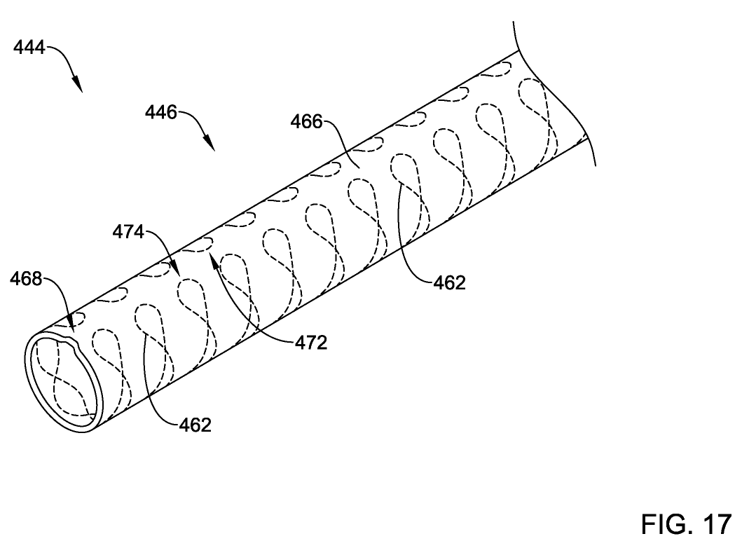

FIG. 17 illustrates another example elongate shaft 444 of the access sheath 40 (shown in FIG. 2). The elongate shaft 444 may be similar in form and function to the elongate shaft 344 described herein. However, FIG. 17 illustrates that the reinforcing wire 462 positioned in a wall 466 of the elongate shaft 444, may include a plurality of crisscrossed regions, whereby the crisscrossed reinforcing wire 362 extends continuously along the longitudinal axis 480 of the elongate shaft 444. Similar to the elongate shaft illustrated and described with respect to FIG. 13, the reinforcing wire 462 may extend only partially about a longitudinal axis 480 (e.g., partially in a circumferential direction about the longitudinal axis 480), whereby the reinforcing wire 462 includes a row of first looped ends 472 circumferentially spaced away from a row of second looped ends 474. In some examples, the crisscrossed reinforcing wire 362 may include axial interconnections whereby a portion of the reinforcing wire 362 is welded or bonded to another portion of the reinforcing wire 362. Like that described herein with respect to the elongate shaft 344, FIG. 17 illustrates that a portion of the wall 466 of the elongate sheath 444 may be tapered about the circumference of the elongate shaft 444 to form a reduced thickness region of the wall 466 which defines a preferential separation region 468.

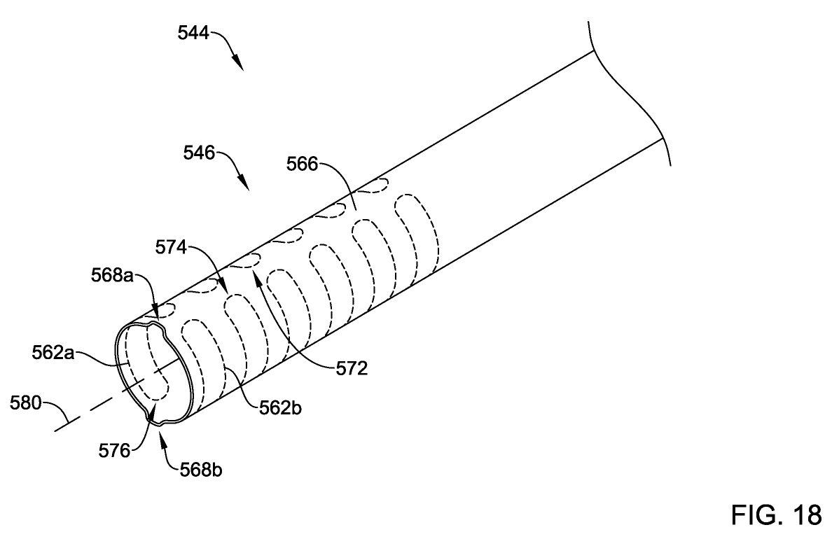

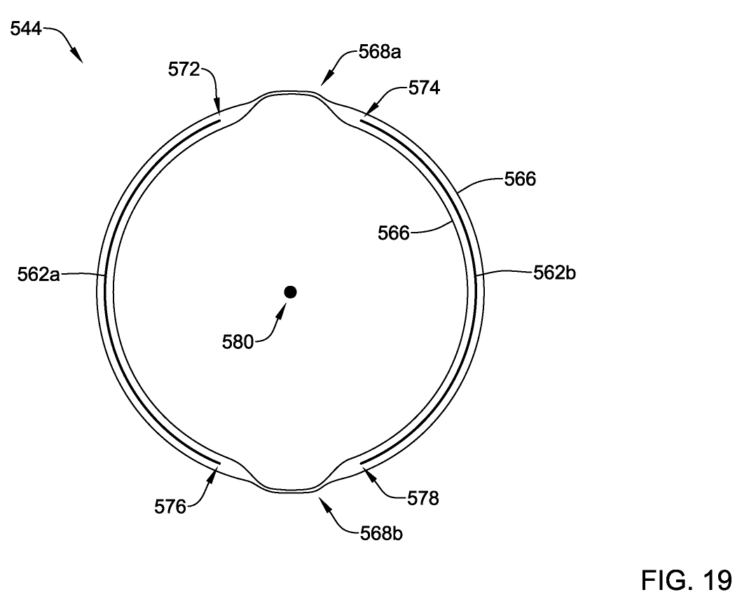

FIG. 18 illustrates another example elongate shaft 544 of the access sheath 40 (shown in FIG. 2). The elongate shaft 544 may be similar in form and function to the elongate shaft 344 described herein. However, FIG. 18 illustrates that the elongate shaft 544 may include a first reinforcing wire 562a and a second reinforcing wire 562b positioned in a wall 566 of the elongate shaft 544, whereby each of the first reinforcing wire 562a and the second reinforcing wire 562b extend continuously along the elongate shaft 544 from the proximal end region to the distal end region 546 of the elongate shaft 544. FIG. 18 further illustrates that each of the first reinforcing wire 562a and the second reinforcing wire 562b may extend only partially about a longitudinal axis 580 (e.g., partially in a circumferential direction about the longitudinal axis 580), whereby the first reinforcing wire 562a includes a includes a row of first looped ends 572 and second loop ends 576 and the second reinforcing wire 562a includes a includes a row of first looped ends 574 and second loop ends 578 (shown in FIG. 19).

In some examples, the wall 566 of the elongate shaft 344 may be formed from one or more layers. For example, the elongate shaft 344 may include a lubricious inner layer or inner liner. In some examples, the inner liner may be etched polytetrafluoroethylene (PTFE) which may be thermally bonded to an outer polymer layer with reinforcing wires positioned in-between. In other examples, an inner liner may have axial perforations for splitting relief, or may be axially devoid of PTFE such that PTFE axial strips extend under reinforcing members (e.g., reinforcing wires). In other examples, the inner liner may grooved for lubricity. In other examples, lubricious elements may be inherently compounded in polymer resins used to form the wall 366. In yet other examples, the wall 366 of the elongate shaft may be partially formed from and/or be coated with a hydrophilic or hydrophobic lubricious coating.

FIG. 19 illustrates a cross-sectional view of the example elongate shaft 544. FIG. 19 illustrates that each of the reinforcing wires 562a, 562b may extend partially around the longitudinal axis 580 of the elongate shaft 544. For example, FIG. 19 illustrates that the first reinforcing wire 562a and the second reinforcing wire 562b are configured such that the ends of the row of first looped ends 572 of the first reinforcing wire 562a is spaced away from the ends of the row of first looped ends 574 of the second reinforcing wire 562b and the ends of the row of second looped ends 576 of the first reinforcing wire 562a is spaced away from the ends of the row of second looped ends 578 of the second reinforcing wire 562b.

Like that described herein with respect to the elongate shaft 344, FIG. 19 illustrates that a portion of the wall 566 of the elongate sheath 544 may be tapered about the circumference of the elongate shaft 544 to form a reduced thickness region of the wall 566 which defines a preferential separation region 568a and a preferential separation region 568b. Further, FIG. 19 illustrates the reduced thickness region of the wall 566 which defines the preferential separation region 568a positioned between the row of first looped ends 572 and the row of first looped ends 574 and the reduced thickness region of the wall 566 which defines the preferential separation region 568b positioned between the row of second looped ends 576 and the row of second looped ends 578. As discussed herein, the preferential separation regions 568a, 568b may define longitudinally extending regions of the elongate shaft 544 which are configured to split, tear, rip in response to a pull-away force being applied to the elongate shaft 544.

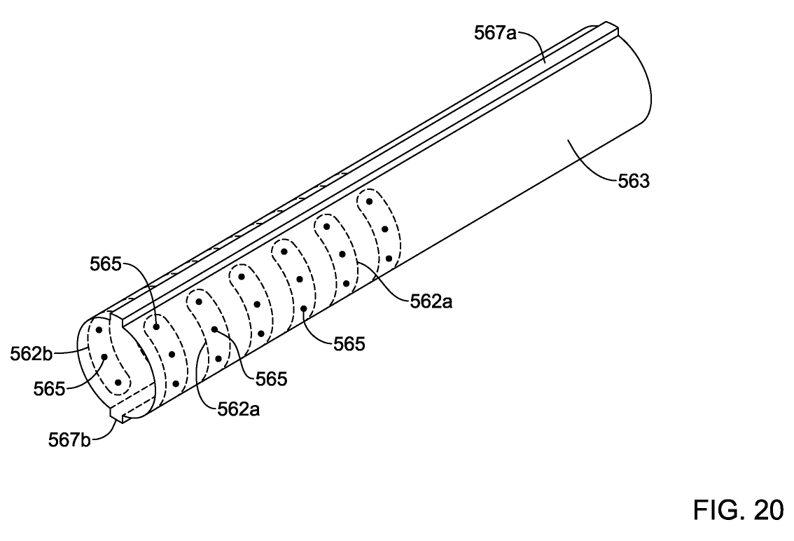

FIG. 20 illustrates an example manufacturing step for constructing the elongate sheath 544 described herein. FIG. 20 illustrates that the reinforcing wires 562a, 562b may be wound along a mandrel 563, whereby the mandrel 563 includes a plurality of pins 565 arranged to form the continuously wound reinforcing wires 562a, 562b illustrated in FIG. 18. It can be appreciated that the arrangement of the pins 565 configures the reinforcing wires 562a, 562b to extend only partially about the longitudinal axis 580 (e.g., partially in a circumferential direction about the longitudinal axis 580) and form the row of first looped ends 572 spaced apart from the row of first looped ends 574 and the row of second looped ends 576 spaced apart from the row of second looped ends 578 as illustrated in FIG. 18. Further, it can be appreciated that that mandrel 563 may include a first spine 567a that permits the formation of the reduced thickness region of the wall 566 which defines the preferential separation region 568a, whereby the preferential separation region 568a is positioned between the row of first looped ends 572 and the row of first looped ends 574. Further, it can be appreciated that that mandrel 563 may include a second spine 567b that permits the formation of the reduced thickness region of the wall 566 which defines the preferential separation region 568b, whereby the preferential separation region 568b is positioned between the row of second looped ends 576 and the row of second looped ends 578. It can be appreciated that a polymer material may be applied to the assembly illustrated in FIG. 20 to form the wall 566 (including the reinforcing wires 562a, 562b positioned therein). The mandrel 563 may then be removed to form the elongate shaft 544 described herein.

FIG. 21 illustrates the splitting of the first hub member 36a and the second hub member 36b of the hub 42 (shown in FIG. 2) and splitting (e.g., peeling, separating) the shaft 44 along both a first frangible region 39a (such as the frangible regions 68, 168a, 368, 468, 568a described herein) and the a second frangible region 39b (such as the frangible region 168b, 568b described herein). It can be appreciated that splitting the shaft 44 along both the first frangible region 39a and the second frangible region 39b may split the shaft 44 into a first shaft portion 35a and a second shaft portion 35b. Further, it can be appreciated that the hub seam 37a (shown in FIG. 2) and the hub seam 37b (hidden from view in FIG. 2), providing a preferential separation region along which the hub 42 may separate, may circumferentially align with the frangible regions 39a, 39b, respectively, of the shaft 44. Accordingly, it can be further appreciated that splitting the shaft 44 may occur along the frangible regions 39a, 39b of the shaft 44, thereby permitting the access sheath 40 to be removed from a guidewire or other portion of a medical device such as the proximal catheter portion 16 of the blood pump 10 shown in FIG. 1.

In some examples, access sheath 40 (or other access sheaths and components thereof described herein) may be made from materials such as metals, metal alloys, polymers, ceramics, metal-polymer composites, or other suitable materials, and the like. Some examples of suitable materials may include metallic materials such as stainless steels (e.g. 304v stainless steel or 316L stainless steel), nickel-titanium alloys (e.g., nitinol, such as super elastic or linear elastic nitinol), nickel-chromium alloys, nickel-chromium-iron alloys, cobalt alloys, nickel, titanium, platinum, or alternatively, a polymeric material, such as a high performance polymer, or other suitable materials, and the like. The word nitinol was coined by a group of researchers at the United States Naval Ordinance Laboratory (NOL) who were the first to observe the shape memory behavior of this material. The word nitinol is an acronym including the chemical symbol for nickel (Ni), the chemical symbol for titanium (Ti), and an acronym identifying the Naval Ordinance Laboratory (NOL).

In some examples, the access sheath 40 (or other access sheaths and components thereof described herein) may be made from materials such as, for example, a polymeric material, a ceramic, a metal, a metal alloy, a metal-polymer composite, or the like. Examples of suitable polymers may include nylons, acrylonitrile butadiene styrene (ABS), polyurethane, a polyether-ester such as ARNITEL® available from DSM Engineering Plastics, a polyester such as HYTREL® available from DuPont, a linear low density polyethylene such as REXELL®, a polyamide such as DURETHAN® available from Bayer or CRISTAMID® available from Elf Atochem, an elastomeric polyamide, a block polyamide/ether, a polyether block amide such as PEBA available under the trade name PEBAX®, silicones, polyethylene, Marlex high-density polyethylene, polyetheretherketone (PEEK), polyimide (PI), and polyetherimide (PEI), a liquid crystal polymer (LCP) alone or blended with other materials. In some examples, a suitable polymeric material may have a yield strain of at least 20%, at least 30%, at least 40%, at least 50%, or more. In some examples, the sheath, the membrane, the liner or other components of the access sheaths disclosed herein may be made from a material having a low coefficient of friction. In some examples, the sheath, the membrane, the liner or other components of the access sheaths disclosed herein may be formed from a fluoropolymer, such as polytetrafluoroethylene (PTFE) or fluorinated ethylene propylene (FEP).

Portions of access sheath 40 (or other access sheaths and components thereof described herein) may be made of, may be doped with, may include a layer of, or otherwise may include a radiopaque material. Radiopaque materials are understood to be materials capable of producing a relatively bright image on a fluoroscopy screen or another imaging technique such as X-ray during a medical procedure. This relatively bright image aids the user of device in determining its location. For example, one or more of the elements described above (i.e., the sheath, the membrane, the medical device, etc.) may include or be formed from a radiopaque material. Suitable materials can include, but are not limited to, bismuth subcarbonate, iodine, gold, platinum, palladium, tantalum, tungsten or tungsten alloy, and the like.

It should be understood that although the above discussion was focused on percutaneous medical procedures within the vasculature of a patient, other examples or methods in accordance with the disclosure can be adapted and configured for use in other parts of the anatomy of a patient. For example, devices and methods in accordance with the disclosure can be adapted for use in the digestive or gastrointestinal tract, such as in the mouth, throat, small and large intestine, colon, rectum, and the like. For another example, devices and methods can be adapted and configured for use within the respiratory tract, such as in the mouth, nose, throat, bronchial passages, nasal passages, lungs, and the like. Similarly, the devices and methods described herein with respect to percutaneous deployment may be used in other types of surgical procedures as appropriate. For example, in some examples, the devices may be deployed in a non-percutaneous procedure. Devices and methods in accordance with the disclosure can also be adapted and configured for other uses within the anatomy.

It should be understood that this disclosure is, in many respects, only illustrative. Changes may be made in details, particularly in matters of shape, size, and arrangement of steps without exceeding the scope of the disclosure. The invention’s scope is, of course, defined in the language in which the appended claims are expressed.

What is claimed is:

1. An access sheath for a percutaneous circulatory support device, comprising:
an inner liner,
a plurality of support members coupled to the inner liner, wherein each support member of the plurality of support members extends at least partially around the circumference of the inner liner, and wherein each support member of the plurality of support members is spaced from one another along a longitudinal axis of the access sheath; and
an outer layer attached to at least a portion of each support member of the plurality of support members.

2. The access sheath of claim 1, wherein each support member of the plurality of support members includes a first end and a second end, and wherein the first end is longitudinally offset from the second end.

3. The access sheath of claim 2, wherein the first end and the second end of each respective support member are circumferentially spaced from one another to define a first preferential separation region extending along the longitudinal axis, and wherein the access sheath is configured to separate along the first preferential separation region.

4. The access sheath of claim 3, wherein the first preferential separation region includes a plurality of perforations, and wherein the plurality of perforations extend along the longitudinal axis of the access sheath.

5. The access sheath of claim 4, wherein at least a portion of each of the plurality of perforations extends radially inward along the first preferential separation region.

6. The access sheath of claim 5, wherein each support member of the plurality of support members is configured to shift from a first position to an expanded position.

7. The access sheath of claim 6, wherein each of the support member of the plurality of support members is configured to self-bias to the expanded position.

8. The access sheath of claim 6, wherein the support members sequentially expand to facilitate splitting of the sheath along the first preferential separation region.

9. The access sheath of claim 4, wherein the outer layer includes a second preferential separation region circumferentially offset from the first preferential separation region.

10. The access sheath of claim 9, wherein the second preferential separation region is circumferentially offset 180 degrees from the first preferential separation region.

11. The access sheath of claim 1, wherein each support member of the plurality of support members is positioned between the inner liner and the outer layer.

12. The access sheath of claim 1, wherein at least a portion of the outer layer is coupled to the inner liner.

13. An access sheath for a percutaneous circulatory support device, comprising:
an inner liner including an outer surface;
a support wire coupled to the inner liner, wherein the support wire extends at least partially around the outer surface of the inner liner; and
an outer layer attached to at least a portion of the support wire.

14. The access sheath of claim 13, wherein the support wire extends continuously along the outer surface of the inner liner.

15. The access sheath of claim 14, wherein the support wire crosses over itself along at least a portion of the inner liner.

16. The access sheath of claim 13, wherein the support wire includes a first plurality of curved end loops extending along a longitudinal axis of the access sheath, a second plurality of curved end loops extending along the longitudinal axis of the access sheath, and wherein the first plurality of curved end loops are radially spaced from the second plurality of curved end loops to define a preferential separation region between the first plurality of curved end loops and the second plurality of curved end loops extending along the longitudinal axis of the access sheath.

17. The access sheath of claim 16, wherein the preferential separation region includes a first wall thickness and the outer layer includes second wall thickness, and wherein the first wall thickness is less than the second wall thickness.

18. The access sheath of claim 13, wherein the support wire is positioned between the inner liner and the outer layer.

19. An access sheath for a percutaneous circulatory support device, comprising:
an inner liner;
a plurality of radial support members coupled to the inner liner, wherein each radial support member of the plurality of radial support members extends at least partially around the circumference of the inner liner, and wherein each support member of the plurality of support members is spaced from one another along a longitudinal axis of the access sheath;
a longitudinal support member extending at least partially along a longitudinal axis of the access sheath; and
an outer layer attached to at least a portion of each radial support members of the plurality of support members and the longitudinal support member.

20. The access sheath of claim 19, wherein each radial support member and the longitudinal support member are positioned between the inner liner and the outer layer.
